(19) United States
(12) Patent Application Publication
(10) Pub. No.: US 2021/0391086 A1
(43) Pub. Date: Dec. 16, 2021

(54) Latent Factor Structuring of Psychopathology

(71) Applicant: X Development LLC, Mountain View, CA (US)

(72) Inventors: Vladimir Miskovic, Binghamton, NY (US); Katherine Elise Link, Palo Alto, CA (US); Nina Thigpen, Sunnyvale, CA (US); Mustafa Ispir, Mountain View, CA (US); Garrett Raymond Honke, Mountain View, CA (US); Pramod Gupta, Mountain View, CA (US)

(21) Appl. No.: 16/897,981

(22) Filed: Jun. 10, 2020

(51) Int. Cl. G16H 50/70; G16H 10/20; G16H 50/20; G16H 10/60; G16H 70/60; G16H 15/00; G06F 16/901; A61B 5/16

(52) U.S. Cl. CPC G16H 50/70; G16H 10/20; G16H 50/20; A61B 5/165; G16H 70/60; G16H 15/00; G06F 16/9024; G16H 10/60

(57) ABSTRACT

Methods, systems, and apparatus, including computer programs encoded on a computer storage medium, for obtaining psychological data, generating a psychopathological analysis data structure (PADS), applying a latent factor analysis algorithm to the PADS to obtain a psychopathological latent factor space (PLFS), generating a latent factor graph, and outputting the latent factor graph.

### TECHNICAL FIELD

This disclosure generally relates reducing psychopathological disorders into a set of latent factors common to a variety of different disorders.

### BACKGROUND

Psychiatric disorders, like major depressive disorder (MDD), are extremely heterogeneous and not well circumscribed by their broad diagnostic labels. For example, the MDD consists of a little over 1,000 unique symptom profiles; an unworkable number by a human alone.

### SUMMARY

In general, the disclosure relates to a process of reducing complex psychopathological disorders into a set of latent factors common to a variety of different disorders. The method uses numerical means to generate a representation of the underlying basis factors of psychopathology, revealing the underlying ‘disorder factors’. For example, the psychological data from a mixture of subjective testing batteries (e.g., PHQ-9, QIDS, BDI-II, etc.) performed on many different users are combined into a psychopathological analysis data structure PADS. The PADS permits the use of a computer to perform latent factor analysis algorithms such as exploratory factor analysis, principal component analysis (PCA), or spectral clustering on the psychological data in order to reduce a broad base of potential psychological disorder symptoms to a more restricted (and more workable) set of underlying causal mechanisms, referred to as psychopathological basis factors or disorder factors.

Psychiatric disorders, like major depressive disorder (MDD), are extremely heterogeneous and not well circumscribed by their broad diagnostic labels. For example, the MDD includes a little over 1,000 unique symptom profiles; an unworkable number by a human alone. However, by reducing these symptom profiles to psychopathological basis factors (e.g., disorder factors), a user can be assessed using a battery of objective (e.g. heart rate, sleep patterns) and subjective (e.g. answers to sense of self, mood, appetite) measurements, a single or multiple times, and assigned a score for each factor. A clinician may use these scores as a basis of treatment, to enable precision psychiatry, and give the clinician tools with which to proceed with evaluation and treatment.

In general, innovative aspects of the subject matter described in this specification can be embodied in methods that include the actions of obtaining psychological data including responses from a plurality of users to a plurality of different psychological assessment batteries, each response being associated with an anonymized user identifier, an assessment battery, and a prompt from the respective assessment battery; generating, using the psychological data, a psychopathological analysis data structure (PADS) by arranging the responses into a data matrix including a first dimension representing anonymized user identifiers, a second dimension representing assessment batteries, and a third dimension representing prompts; applying a latent factor analysis algorithm to the PADS to obtain a psychopathological latent factor space (PLFS) representing a reduced set of basis factors common to multiple psychopathological disorders; generating, from the reduced set of basis factors, a latent factor graph including a first set of nodes representing the basis factors linked, by respective edges, to a second set of nodes representing the psychopathological disorders and outputting the latent factor graph. Other implementations of this aspect include corresponding systems, apparatus, and computer programs, configured to perform the actions of the methods, encoded on computer storage devices.

These and other implementations can each optionally include one or more of the following features.

In some implementations, the psychological assessment batteries can include one or more of PHQ-9, QIDS, BDI-II, and Ecological Momentary Assessment questionnaires.

In some implementations, the latent factor analysis algorithm can include one of an exploratory factor analysis algorithm, a principal component analysis algorithm, or a spectral clustering algorithm.

In some implementations, a subset of the patient identifiers can include patient identifiers that are associated with pre-diagnosed psychopathological disorders.

In some implementations, the PLFS can further identify correlations between the basis factors and the psychopathological disorders.

In some implementations, edge thickness in the latent factor graph can represent a correlation strength between a respective basis factor and a respective psychopathological disorder.

In some implementations, a subset of the patient identifiers can include patient identifiers that are associated with pre-diagnosed psychopathological disorders, and at least one patient identifier is associated with a patient who has not yet been diagnosed with a psychopathological disorder.

In some implementations, a subset of the patient identifiers can include patient identifiers that are associated with pre-diagnosed psychopathological disorders, and at least one patient identifier is associated with a patient who has not yet been diagnosed with a psychopathological disorder.

In some implementations, the PFLS can further determine one or more potential diagnoses for the at least one patient identifier.

In some implementations, the PFLS can further represent at least one patient identifier as a node on the latent factor graph.

In some implementations, the psychological assessment batteries can include one or more batteries of objective patient data.

Particular implementations of the subject matter described in this specification can be implemented so as to realize one or more of the following technical advantages. Implementations provide unique data structure that permits a computationally based analysis of distinct features from across multiple disparate psychopathology diagnosis batteries. Implementations permit the application of machine learning techniques to distinct and disparate diagnosis batteries. For example, the psychopathological analysis data structure (PADS), described below, permits latent factor analysis computations to be conducted by computer systems on a wide variety of diagnosis batters to identify latent factors common to multiple different psychological disorders.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts a block diagram of an example system for determining the latent psychopathology factors.

FIGS. 2A and 2B depict graphical representations of a psychopathological analysis data structure (PADS).

FIGS. 3A and 3B depict example latent factor graph outputs.

FIG. 4 depicts a flowchart of an example process for determining the latent factor space of psychopathology in accordance with implementations of the present disclosure.

FIG. 5 depicts a schematic diagram of a computer system that may be applied to any of the computer-implemented methods and other techniques described herein.

Like reference numbers and designations in the various drawings indicate like elements.

### DETAILED DESCRIPTION

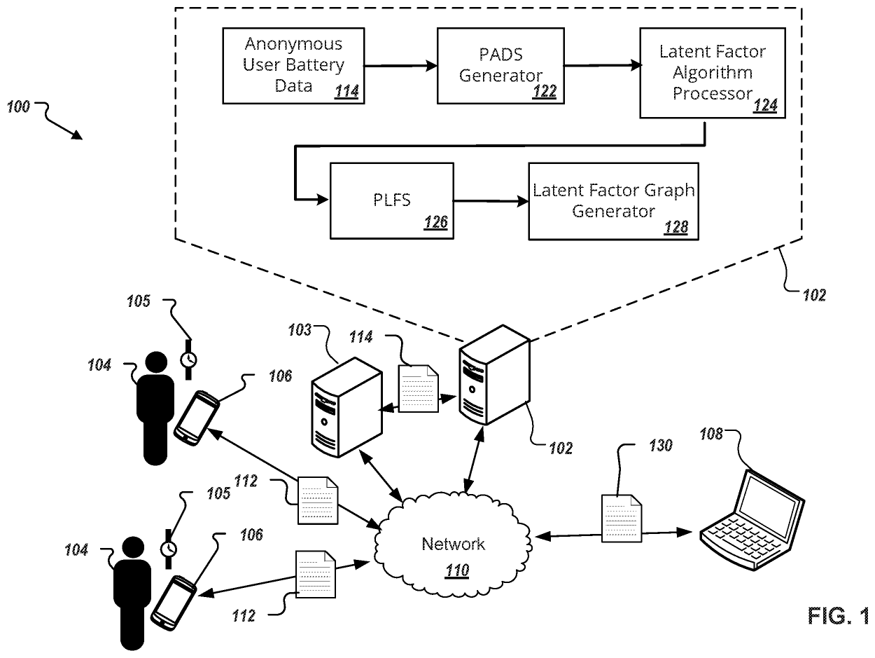

FIG. 1 is a diagram that illustrates an example of a system 100 for determining the latent factors of psychopathology. The system 100 includes a psychopathology analysis system (PAS) 102 and a receiving system 103 in communication with a plurality user computing devices 106, 108 over a network 110. Network 110 can include public and/or private networks and can include the Internet. In some implementations, the PAS 102 and/or the receiving system 103 are also in communication with one or more wearable devices 105.

The PAS 102 can include a system of one or more computers. In general, the PAS 102 is configured to perform latent factor analysis on psychological data to reduce complex psychopathological disorders into a set of latent factors common to a variety of different disorders. The PAS 102 obtains anonymized psychological data from the receiving system 103. The psychological data can include, but is not limited to, patient responses to subjective testing batteries (e.g., PHQ-9, QIDS, BDI-II, etc.), batteries of objective patient data (e.g. heart rate, sleep patterns, behavioral data from cognitive assessments), or both. The PAS 102 then applies the psychological data as input to a latent factor algorithm to generate a reduced set of basis factors common to multiple psychopathological disorders represented by the different batteries. For example, the PAS 102 can store and execute one or more machine learning engines that are programmed to determine the latent factor structure of psychopathology by performing the processes described below.

The receiving system 103 can include a system of one or more computers. The receiving system 103 can be configured to receive, store, and anonymize patient psychological data. For example, the receiving system 103 is configured to collect and anonymize patient psychological data 112 from computing devices 106 and/or wearable devices 105. For example, the receiving system 103 can obtain patient responses to subjective testing batteries (e.g., PHQ-9, QIDS, BDI-II, etc.) from patient responses to questionnaire batteries completed on computing devices 106. The receiving system 103 can obtain batteries of objective patient data (e.g. heart rate, sleep patterns) from patient wearable devices 105. The receiving system 103 can obtain the psychological data 112 from wearable devices 105, and computing devices 106 as actively submitted by users 104 or passively, e.g., sleep assessment data. The data that the receiving system 103 collects can come in the form of answers to batteries of psychological or physiological tests. For example, a battery can be in the form of a number of specialized cognitive tests, collection of sleep quality data through user wearable devices, psychiatric questionnaires, or ecological momentary assessments (EMAs), or any combination thereof. The user 104 (e.g., patient) may input answers to a battery through computing devices 106 or a computing device 106 at a clinician's office, e.g., desktop computers, laptop computers, smart phones, tablet computers. In some implementations, wearable devices 105 may physiological data from the users 104 (e.g., heart rate, sleep patterns, etc.).

In some implementations, data access rules executed by the receiving system 103 permit the receiving system 103 to obtain the psychological data 112 without third-party human interaction with the data on the receiving system 103, thereby, protecting the patient's privacy. The receiving system 103 further protects each patient's (user 104) privacy by the receiving system 103 assigns anonymized user identifiers to each patient whose data is obtained. The receiving system 103 can use the anonymized user identifiers to correlate data to specific patients while protecting personal information. In some implementations, the receiving system 103 tags the psychological data 112. In some examples, the receiving system 103 can apply tags to the anonymized identifiers that indicate whether or not the associated patient has been previously diagnosed with a psychological disorder and, if so, identify the disorder. In some implementations, patients are permitted to opt-in or opt-out of having their psychological information used by a PAS 102 for latent factor analysis purposes (e.g., research and/or diagnosis).

The anonymized psychological data 114 is passed to the PAS 102. For example, anonymized psychological data 114 for multiple patients can be transmitted to the PAS 102 in bulk. In other words, the receiving system 103 can serve as a repository of anonymized psychological data from a large sample of patients. The receiving system 103 can transmit the anonymized psychological data 114 to the PAS 102 through network 110. In some implementations, the receiving system 103 can transmit the anonymized psychological data 114 to the PAS 102 through a direct (e.g., hardwired) network connection or a secure network connection (e.g., a virtual private network), e.g., to maintain data security.

In some implementations, the functions performed by the receiving system 103 and the PAS 102 can be combined into one server system. In such implementations, however, the data anonymization process can be performed on separate computing devices within the system to maintain privacy through data segregation.

Wearable devices 105 can be wearable computing devices, e.g., smart watches, health tracking devices, smart rings. Computing devices 106, 108 can be computing devices, e.g., mobile phones, smart phones, tablet computers, laptop computers, desktop computers, home assistant devices, or other portable or stationary computing devices. Computing device 108 can be a computing device associated with a clinician (e.g., a psychologist or a psychiatrist) to which the PAS 102 transmits completed latent factor analysis results (e.g., output 130).

In some implementations, the receiving system 103 can permit authorized users (e.g., clinicians) to interact with subsets of the psychological data 112. For example, the receiving system 103 can provide a secure portal for a clinician to select a subset of patient data for analysis by the PAS 102. In such implementations, the clinician can be restricted to interacting only with data related to his or her own patients. Thus, a clinician can configure various latent factor analyses for particular patients or subsets of patients.

In various implementations, PAS 102 can perform some or all of the operations related to performing latent factor analysis on psychological data. For example, PAS 102 can include a PADS generator 122, a latent factor algorithm processor 124, and a latent factor graph generator 128. The PADS generator 122, latent factor algorithm processor 124, and latent factor graph generator 128 can each be provided as one or more computer executable software modules or hardware modules. That is, some or all of the functions of PADS generator 122, latent factor algorithm processor 124, and latent factor graph generator 128 can be provided as a block of code, which upon execution by a processor, causes the processor to perform functions described below. Some or all of the functions of ADS generator 122, latent factor algorithm processor 124, and latent factor graph generator 128 can be implemented in electronic circuitry, e.g., as field programmable gate array (FPGA) or an application specific integrated circuit (ASIC).

PAS 102 can also implement one or more machine learning engines that analyze the anonymized psychological data 114 to generate a psychopathological latent factor space (PLFS) representing a reduced set of basis factors common to multiple psychopathological disorders. For example, latent factor algorithm processor 124 can be implemented as one or more machine learning models. More specifically, PAS 102 includes one or more machine learning models that have been trained to receive model inputs (e.g., anonymized psychological data 114) and to generate a reduced set of basis factors common to multiple psychopathological disorders based on the received model input. In some implementations, the machine learning model executes an exploratory factor analysis algorithm, a principle component analysis algorithm, or a spectral clustering algorithm.

In operation, PAS 102 receives anonymized psychological data 114 from the receiving system 103. The anonymized psychological data 114 can be passed to the PADS generator 122. The anonymized psychological data 114 can include, but is not limited to, anonymized patient responses to various different diagnosis batteries. For example, the psychological assessment batteries can include one or more of PHQ-9, QIDS, BDI-II, Ecological Momentary Assessment questionnaires, and objective physiological measurements of a patient.

The PADS generator 122 arranges the anonymized psychological data 114 into a psychopathological analysis data structure (PADS). A PADS is a data structure that permits a computationally based analysis of distinct features from across multiple disparate psychopathology diagnosis batteries. In other words, PADS generator 122 arranges anonymized psychological data from various distinct and disparate diagnosis batteries into a data structure that is processable by machine learning algorithms to infer a reduced set of basis factors common to multiple psychopathological disorders represented by the different batteries. As will be described in more detail below, the inferred reduced basis factors (e.g., PLFS) can be represented by a latent factor graph to aid clinicians in making more accurate and efficient diagnoses.

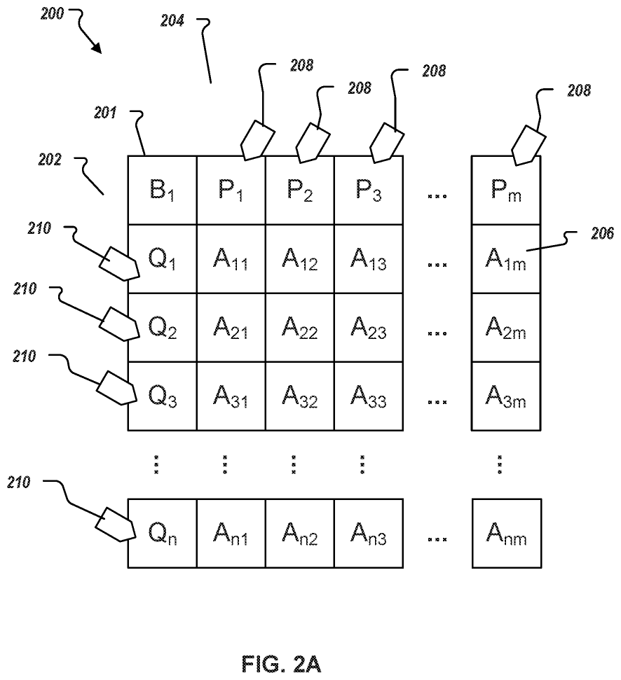

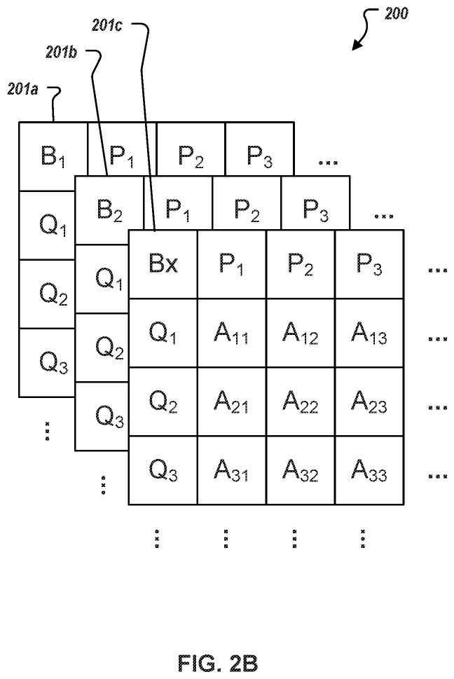

FIGS. 2A and 2B depict graphical representations of a PADS 200. Referring to FIGS. 1, 2A, and 2B, PADS generator 122 arranges patient responses to each diagnosis battery, B₁ through Bₓ, into a battery matrix 201. The PADS generator 122 can separate each battery into a set of prompts and patient responses to the prompts. For some batteries, the prompts are questions from a diagnosis questionnaire and the responses are patient answers to the questions. For other batteries, the prompts can be a time scale, e.g., a series of discrete time periods, and the patient response is a measurement of a particular physiological characteristic of the patient at a time on the time scale. For example, the responses may be a heart rate at particular times of the day.

For each battery, the PADS generator 122 arranges the prompts (Q₁-Qₙ) along a first dimension 202 of the matrix 201 and patient identities (P₁-Pₘ) along a second dimension 204 of the matrix 201. The responses (A₁₁-Aₙₘ) associated with each prompt and patient identity are arranged in corresponding cells 206 of the matrix 201. Prompts and responses associated with each different battery (B₁-Bₓ) are arranged in separate matrices 201a, 201b, 201c along a third dimension (e.g., the z-dimension as depicted graphically in FIG. 2B).

In some implementations, the anonymized psychological data 114 is tagged with additional data. The tags 208, 210, can be metadata that describes various characteristics of information contained in the anonymized psychological data 114. For example, patient identifiers can include tags 208 that indicate an existing or prior diagnosis of the represented patient. In some examples, patient identifier tags 208 can include information including, but not limited to, whether the represented patient has or has not yet been diagnosed, and if so, and indication of the diagnosis (e.g., major depressive disorder, anxiety, etc.). In some examples, prompt identifier tags 210 can include information including, but not limited to, data indicating symptom related to the prompt. That is, the tag 210 can be used to identify one or more particular symptoms or factors that may be indicated by a patient's response to the prompt. For example, responses to a question such as “How motivated do you feel?” can be tagged as being potentially indicative of anhedonia, which itself may be a symptom of either bipolar disorder or depression.

The PAD 200 is provided to the latent factor algorithm processor latent factor algorithm processor 124. The latent factor algorithm processor latent factor algorithm processor 124 can be a server or a network of servers that hosts and executes a latent factor algorithm to process the PADS 200 and reduce the PADS 200 into an underlying set of psychopathological disorder factors. The latent factor algorithm system latent factor algorithm processor 124 can implement one or more factor analysis algorithms including, but not limited to, principal component analysis, spectral clustering, or exploratory factor analysis. The latent factor algorithm processor 124 outputs a psychopathological latent factor space (PLFS) 126. For example, a three dimensional PADS 200 can be processed into a two dimensional PLFS 126. The PLFS 126 serves as a basis function for psychological disorders. In other words, the PLFS 126 forms a set of basis symptoms (e.g., disorder factors) that correlate with a particular psychological disorder.

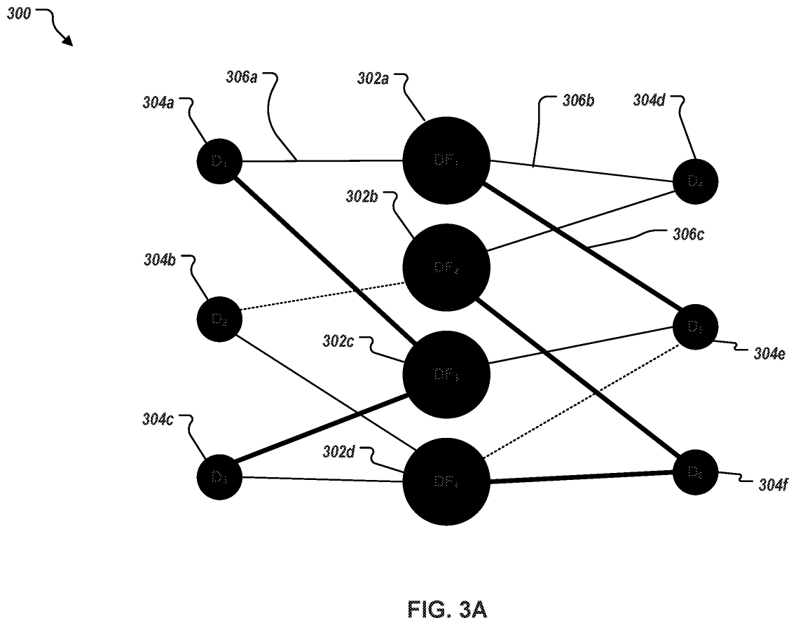

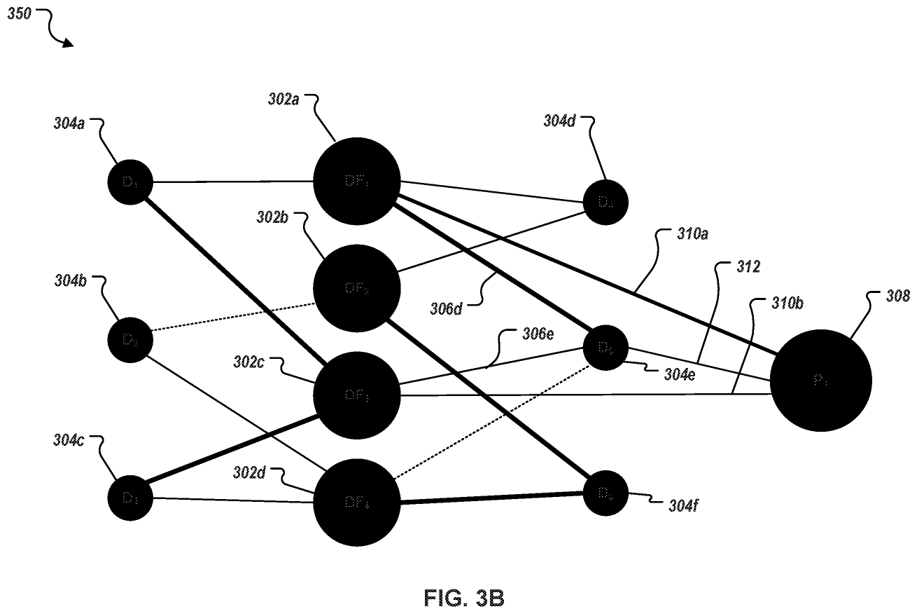

In more detail, the latent factor algorithm processor 124 identifies correlations between various disorder factors, e.g., indicated by the prompts and associated patient responses the PADS 200, with different psychological disorders, e.g., indicated by patient identifier tags 210. For example, the latent factor algorithm processor 124 can employ a latent factor algorithm to identify clusters within the patient response (e.g., A₁₁-Aₙₘ). Based on the identified clusters, the latent factor algorithm processor 124 can use the patient tags 208 and prompt tags 210 to identify correlations between particular disorders and disorder factors (or symptoms). The resulting latent factor space is a PLFS 126 that can be used to generate a latent factor graph (shown in FIGS. 3A and 3B) to graphically illustrate the identified correlations.

The latent factor algorithm processor 124 can be a vector of basis factors, each of which can be termed a disorder factor, connected to one or more diagnosable disorders. For example, the PLFS can indicate correlation strengths, e.g., as represented by correlation scores, between various underlying disorder factors and psychopathological disorders. For instance, anhedonia can be an example of a potential disorder factor. The latent factor algorithm processor 124 may identify correlations between positive responses to prompts related to anhedonia and patients who have been diagnosed with several different psychopathological disorders, e.g., depressive disorders, substance related disorders, psychotic disorders, and personality disorders.

The PLFS 126 is provided to latent factor graph generator 128 within the PAS 102. The latent factor graph generator 128 executes a latent factor graph generation algorithm on the PLFS 126. For example, latent factor graph system 128 parses the PLFS 126 to generate a graphical representation of the basis vector spaces. FIG. 3 depicts an example of a latent factor graph 300. The latent factor graph generator 128 can represent disorder factors identified in the PLFS 126 as disorder factor nodes 302. Each node 302 represents a different disorder factor, e.g., DF₁ 302a, DF₂ 302b, DF₃ 302c, and DF₄ 302d. The latent factor graph generator 128 can represent psychopathological disorders identified in the PLFS 126 as disorder nodes 304. Each node 304 represents a different disorder, e.g., D₁ 304a, D₂ 304b, D₃ 304c, D₄ 304d, D₅ 304e, D₆ 304f, Further, latent factor graph generator 128 can represent correlations between the disorder factors and the psychopathological disorders by edges 306 connecting disorder factor nodes 302 with related psychopathological disorder nodes 304. For example, node 302a (DF1) may represent anhedonia. In such an example, disorder nodes 304a, 304d, and 304e (D1, D4, and D5) would represent psychopathological disorders that are correlated with anhedonia as indicated by edges 306a, 306b, and 306c.

In some implementations, a threshold value can be used to determine which correlations scores are great enough to signify a correlation between a basis factor and a psychopathological disorder. For example, basis factor/psychological disorder pairs may only be represented by an edge in the latent factor graph when the correlation score between the two is greater than or equal to the threshold value.

In some implementations, the relative strength of correlations between a psychopathological disorder 304 and a disorder factor 302 can be represented by a characteristic of the edge 306. Such characteristics can include, but are not limited to, edge thickens and edge color. For example, the latent factor graph 300 uses a heavy connecting line to depict a relatively strong correlation, and a dashed line to depict a relatively weak correlation. For example, disorder D₅ 304e is connected to disorder factor DF₁ 302a by a relatively strong correlation 306c, disorder factor DF₂ 302b by a relatively average correlation 306b, and disorder factor DF₃ 302c by a relatively weak correlation 306c as indicated by the thickness of the edges.

After the latent factor algorithm processor 124 has determined the correlations between psychopathological disorders 304 and disorder factors 302, newly transmitted anonymous user battery data files 114 that are processed into a PADS 200 can be assigned a score in one or more of the disorder factors 302. For example, an anonymous user battery data file 114 correlated with user P₁ 308 can be assigned a score in multiple disorder factors, e.g., scores in disorder factors D₁ 304a and D₃ 304c. The scores in disorder factors DF₁ 304a and DF₃ 304c are depicted in FIG. 3 as connecting lines 310a and 310b. Connecting line 310a depicts a heavier weight than connecting line 310b which can indicate a higher score value. In some implementations, numerical correlation scores can be presented above the respective edges 306.

In some implementations, a PADS 200 containing only patients with diagnoses is stored for use in diagnosing previously undiagnosed patients, e.g., as a diagnosis PADS. For example, in some implementations, the PAS 102 can be used to aid in diagnosing undiagnosed patients by adding one or more undiagnosed patient identifiers to a diagnosis PADS. For example, the PADS generator 122 can add a column of responses associated with each undiagnosed patient identifier to the appropriate battery matrix or matrices. The latent factor algorithm processor 124 can then process the diagnosis PADS with the appended undiagnosed patient identifiers. The latent factor algorithm processor 124 identifies correlations between the responses associated with the undiagnosed patient identifier(s) and disorder factors. From the correlated disorder factors, the latent factor algorithm processor 124 can identify likely psychopathological disorder(s) that each undiagnosed patient identifier may be suffering from. The resulting PLFS 126 can include correlation scores between the undiagnosed patient identifier(s) and the disorder factors, correlation scores between the undiagnosed patient identifier(s) and the psychopathological disorders, or both.

In some implementations, the latent factor algorithm processor 124 can infer a diagnosis for the undiagnosed patient by comparing correlations scores between the undiagnosed patient's responses and a set of disorder factors with correlations between a particular psychopathological disorder and the same (or a similar) set of disorder factors. For example, if the responses of the undiagnosed patient correlate to a set of disorder factors that is similar to those to which a particular psychopathological disorder correlates; and with similar correlation scores, the latent factor algorithm processor 124 can infer that the undiagnosed patient likely suffers from that particular psychopathological disorder. Furthermore, the latent factor algorithm processor 124 can determine a confidence of the diagnosis based on the similarity of the undiagnosed patient's correlation scores to the disorder factors and the psychopathological disorders correlation scores to the same disorder factors. For example, the closer in magnitude the two sets of correlation scores are to each other, the greater the confidence of the diagnosis.

FIG. 3B illustrates an example latent factor graph 350 that includes an undiagnosed patient identifier. For example, latent factor graph generator 128 can represent the undiagnosed patient with a patient node 308 (P₁). As illustrated, responses of the undiagnosed patient (P₁) are correlated with disorder factors DF₁ and DF₃. This is indicated by b edges 310a and 310b connecting the patient node 308 to disease factor nodes 302a and 302c, respectively. Similarly, psychopathological disorder D₅ (node 304e) is also correlated with disorder factors DF₁ (node 302a) and DF₃ (node 302c) as indicated by edges 306d and 306e, respectively. Moreover, the correlation scores between the undiagnosed patients responses and the disorder factors DF₁ and DF₃ is similar to the correlation scores of DF₁ and DF₃ with psychopathological disorder D₅, e.g., as indicated by the relevant edge thicknesses. Thus, the latent factor algorithm processor 124 may infer, based on these similar correlation scores, that the patient is likely suffering from psychopathological disorder D₅. In other words, because DF₁ 304a is strongly connected, and DF₃ 302c is moderately connected with disorder D₅ 304e and because patient P₁ 308 is connected with similar strengths to these same disorder factors, the system can suggest a presumptive diagnosis (e.g., edge 312) for patient P₁ of disorder D₅ 304e as determined by the latent factor analysis algorithm.

Referring again to FIG. 1, after a latent factor graph 128 has been generated, it can be transmitted via a network to a clinician device 106 for viewing and diagnosis. The clinician device 106 can also transmit latent factor graphs 128 to the receiving system 103 which can then distribute the latent factor graphs 128 to user screen 106 and wearable devices 105.

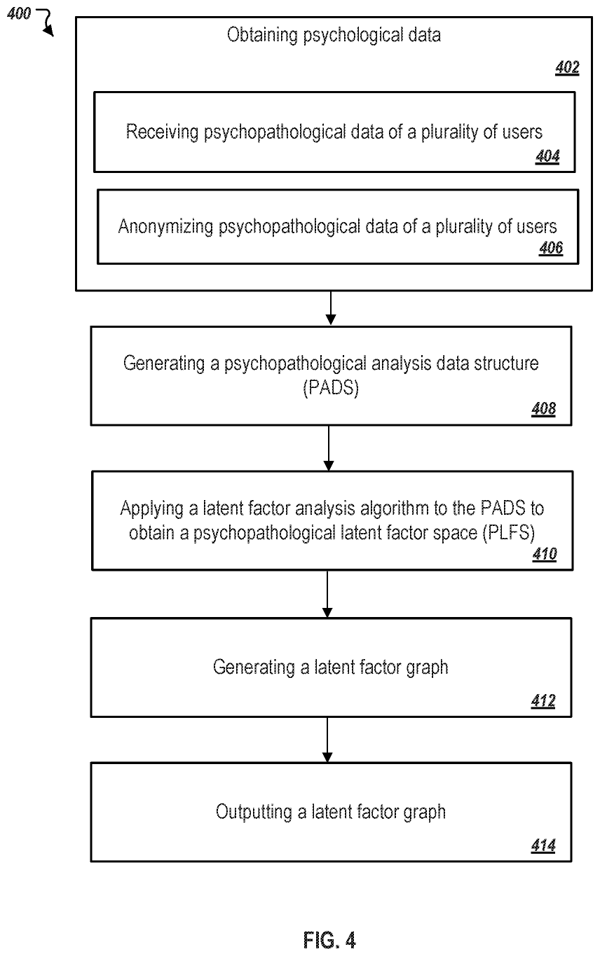

FIG. 4 depicts a flowchart of an example process 400 for determining the latent factors of psychopathology in accordance with implementations of the present disclosure. In some implementations, the process 400 can be provided as one or more computer-executable programs executed using one or more computing devices such as PAS 102 and receiving system 103. In some examples, process 400 is executed using one or more machine learning models.

The system obtains user psychopathological data (402). In some examples, the system receives psychopathological data from a plurality of users (404). For example, patient responses to subjective and objective testing batteries can be received by and stored by a server system (e.g., receiving server system 103 or PAS 102). The psychopathological data can include, but is not limited to, patient responses to subjective testing batteries (e.g., PHQ-9, QIDS, BDI-II, etc.) and objective patient data (e.g. heart rate, sleep patterns) from patient wearable devices. Furthermore each test battery in the psychopathological data can include a set of prompts and patient responses to the prompts. For some batteries, the prompts are questions from a diagnosis questionnaire and the responses are patient answers to the questions. For other batteries, the prompts can be a time scale, e.g., a series of discrete time periods, and the patient response is a measurement of a particular physiological characteristic of the patient at a time on the time scale. For example, the responses may be a heart rate at particular times of the day.

In some implementations, the psychopathological data is tagged to indicate attributes of the data. For example, the psychopathological data can be tagged with metadata tags indicating previous diagnoses of patients. As another example, prompts can be tagged with data indicating particular symptom(s) targeted by the prompt. That is, a prompt data tag can indicate which symptom(s) would be indicated by a positive patient response to the prompt. For example, a prompt such as “How motivated are you to engage the world?” can be tagged as being potentially indicative of anhedonia.

The system anonymizes the psychopathological data. For example, the system can remove personally identifiable information and assign a unique patient identifier to each unique patient. In some examples, the patient identifiers may be non-reversible to protect each patient's identity. In some examples, the system can perform a cryptographic hash function on particular aspects of each patient's identity, e.g., the system can hash a combination of the patient's name, address, and date of birth to obtain a unique patient identifier.

The system generates a PADS (408). For example, the system can arrange anonymized psychological data from various distinct and disparate diagnosis batteries into a data structure that is processable by machine learning algorithms to infer a reduced set of basis factors common to multiple psychopathological disorders represented by the different batteries. For example, the system can arrange the psychopathological data into a data matrix with a first dimension representing anonymized user identifiers, a second dimension representing assessment batteries, and a third dimension representing prompts.

The system applies a latent factor analysis algorithm to the PADS to obtain a PLFS (410). For example, the system can process the PADS by applying one or more latent factor analysis algorithms including principal component analysis, spectral clustering, or exploratory factor analysis. The PLFS can be a latent vector space of one or more disorder factors. For example, the PLFS can indicate correlation strengths, e.g., as represented by correlation scores, between various underlying disorder factors and psychopathological disorders. For instance, anhedonia is a deficit in the ability to feel pleasure and is listed as a component of depressive disorders, substance related disorders, psychotic disorders, and personality disorders. As an example, a PLFS may exhibit correlations between anhedonia (as a disorder factor) and depressive disorders, substance related disorders, psychotic disorders, and personality disorders.

The system generates a latent factor graph (412) and outputs the latent factor graph for presentation to a user (e.g., a clinician) (414). For example, the system can execute a latent factor graph generation algorithm on the PLFS. The latent factor graph is a graphical depiction of the PLFS. For example, the PLFS can include a first set of nodes representing the disorder factors (e.g., basis factors of the PLFS) linked, by respective edges, to a second set of nodes representing the psychopathological disorders. The edges can be determined based on correlations scores between the basis factors and the psychopathological disorders. Moreover, the magnitude of each correlation score can be represented by characteristics of the edge, e.g., a color, thickness, line type, etc.

In some implementations, a threshold value can be used to determine which correlations scores are great enough to signify a correlation between a basis factor and a psychopathological disorder. For example, basis factor/psychological disorder pairs may only be represented by an edge in the latent factor graph when the correlation score between the two is greater than or equal to the threshold value.

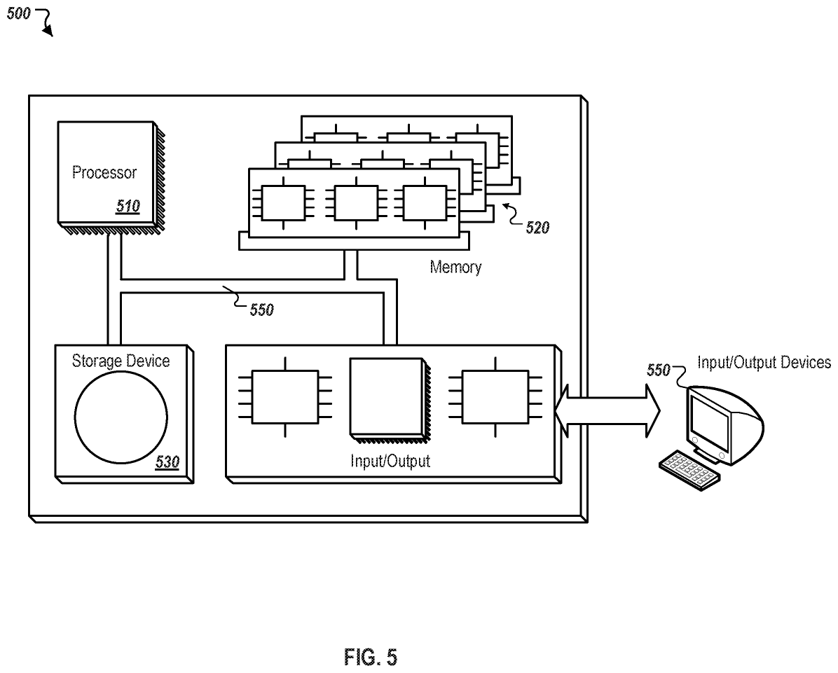

FIG. 5 is a schematic diagram of a computer system 500. The system 500 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 500) and their structural equivalents, or in combinations of one or more of them. The system 500 is intended to include various forms of digital computers, such as laptops, desktops, workstations, personal digital assistants, servers, blade servers, mainframes, and other appropriate computers, including vehicles installed on base units or pod units of modular vehicles. The system 500 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

For convenience, implementations of the present disclosure have been discussed in further detail with reference to an example medical context. More specifically, the example context includes predicting the spread of a contagion (e.g., an illness). It is appreciated, however, that implementations of the present disclosure can be realized in other appropriate contexts (e.g., predicting the spread of ideas, social trends, word-of-mouth advertising, etc.).

What is claimed is:

1. A computer-implemented psychopathology latent factor analysis method executed by one or more processors, the method comprising:
obtaining psychological data comprising responses from a plurality of users to a plurality of different psychological assessment batteries, each response being associated with an anonymized user identifier, an assessment battery, and a prompt from the respective assessment battery;
generating, using the psychological data, a psychopathological analysis data structure (PADS) by arranging the responses into a data matrix comprising a first dimension representing anonymized user identifiers, a second dimension representing assessment batteries, and a third dimension representing prompts;
applying a latent factor analysis algorithm to the PADS to obtain a psychopathological latent factor space (PLFS) representing a reduced set of basis factors common to multiple psychopathological disorders;
generating, from the reduced set of basis factors, a latent factor graph comprising a first set of nodes representing the basis factors linked, by respective edges, to a second set of nodes representing the psychopathological disorders; and
outputting the latent factor graph.

2. The method of claim 1, wherein the psychological assessment batteries include one or more of PHQ-9, QIDS, BDI-II, and Ecological Momentary Assessment questionnaires.

3. The method of claim 1, wherein the latent factor analysis algorithm includes one of an exploratory factor analysis algorithm, a principal component analysis algorithm, or a spectral clustering algorithm.

4. The method of claim 1, wherein a subset of the patient identifiers include patient identifiers that are associated with pre-diagnosed psychopathological disorders.

5. The method of claim 4, wherein the PLFS identifies correlations between the basis factors and the psychopathological disorders.

6. The method of claim 1, wherein a thickness of edges in the latent factor graph represents a correlation strength between a respective basis factor and a respective psychopathological disorder.

7. The method of claim 1, wherein a subset of the patient identifiers include patient identifiers that are associated with pre-diagnosed psychopathological disorders, and at least one patient identifier is associated with a patient who has not yet been diagnosed with a psychopathological disorder.

8. The method of claim 7 further comprising determining, from the PLFS, one or more potential diagnoses for the at least one patient identifier.

9. The method of claim 8 further comprising representing the at least one patient identifier as a node on the latent factor graph.

10. The method of claim 1, wherein the psychological assessment batteries comprise one or more batteries of objective patient data.

11. A system comprising:
at least one processor; and a data store coupled to the at least one processor having instructions stored thereon which, when executed by the at least one processor, causes the at least one processor to perform operations comprising:
generating, using the psychological data of claim 1, a psychopathological analysis data structure (PADS) by arranging the responses into a data matrix comprising a first dimension representing anonymized user identifiers, a second dimension representing assessment batteries, and a third dimension representing prompts;
applying a latent factor analysis algorithm to the PADS to obtain a psychopathological latent factor space (PLFS) representing a reduced set of basis factors common to multiple psychopathological disorders;
generating, from the reduced set of basis factors, a latent factor graph comprising a first set of nodes representing the basis factors linked, by respective edges, to a second set of nodes representing the psychopathological disorders; and
outputting the latent factor graph.

12. The system of claim 11, wherein the latent factor analysis algorithm includes one of an exploratory factor analysis algorithm, a principal component analysis algorithm, or a spectral clustering algorithm.

13. The system of claim 11, wherein the PLFS identifies correlations between the basis factors and the psychopathological disorders.

14. The system of claim 11, wherein a thickness of edges in the latent factor graph represents a correlation strength between a respective basis factor and a respective psychopathological disorder.

15. The system of claim 11, wherein a subset of the patient identifiers include patient identifiers that are associated with pre-diagnosed psychopathological disorders, and at least one patient identifier is associated with a patient who has not yet been diagnosed with a psychopathological disorder.

16. A non-transitory computer readable storage device storing instructions that, when executed by at least one processor, cause the at least one processor to perform operations comprising:
generating, using the psychological data of claim 1, a psychopathological analysis data structure (PADS) by arranging the responses into a data matrix comprising a first dimension representing anonymized user identifiers, a second dimension representing assessment batteries, and a third dimension representing prompts;
applying a latent factor analysis algorithm to the PADS to obtain a psychopathological latent factor space (PLFS) representing a reduced set of basis factors common to multiple psychopathological disorders;
generating, from the reduced set of basis factors, a latent factor graph comprising a first set of nodes representing the basis factors linked, by respective edges, to a second set of nodes representing the psychopathological disorders; and
outputting the latent factor graph.

17. The method of claim 16, wherein the latent factor analysis algorithm includes one of an exploratory factor analysis algorithm, a principal component analysis algorithm, or a spectral clustering algorithm.

18. The method of claim 16, wherein the PLFS identifies correlations between the basis factors and the psychopathological disorders.

19. The method of claim 16, wherein a thickness of edges in the latent factor graph represents a correlation strength between a respective basis factor and a respective psychopathological disorder.

20. The method of claim 16, wherein a subset of the patient identifiers include patient identifiers that are associated with pre-diagnosed psychopathological disorders, and at least one patient identifier is associated with a patient who has not yet been diagnosed with a psychopathological disorder.
